(19) United States
(12) Patent Application Publication
(10) Pub. No.: US 2021/0363486 A1
(43) Pub. Date: Nov. 25, 2021

(54) CARDIOMYOCYTE PROLIFERATION PROMOTING AGENT AND USE THEREOF

(71) Applicant: KYOTO UNIVERSITY, Kyoto-shi, Kyoto (JP)

(72) Inventors: Yoshinori YOSHIDA, Kyoto-shi, Kyoto (JP); Takeshi HATANI, Kyoto-shi, Kyoto (JP); Manabu KASAMOTO, Kyoto-shi, Kyoto (JP); Shunsuke FUNAKOSHI, Kyoto-shi, Kyoto (JP)

(21) Appl. No.: 16/968,125

(22) PCT Filed: Feb. 8, 2019
(86) PCT No.: PCT/JP2019/004631

§ 371 Date: Aug. 6, 2020

(30) Foreign Application Priority Data
Feb. 9, 2018 (JP) 2018-021729

(51) Int. Cl. C12N 5/077; G01N 33/50

(52) U.S. Cl. CPC C12N 5/0657; C12N 2501/999; G01N 33/5061

(57) ABSTRACT

By using a drug that activates the cell cycle, including a retinoic acid receptor agonist, a phosphatidylinositol 3-kinase (PI3K) inhibiting agent, or an isocitrate dehydrogenase 1 (IDH1) inhibiting agent, cardiomyocytes can be made to propagate efficiently, and engraftment rate of cardiomyocytes can also be increased at the time of transplantation.

### TECHNICAL FIELD

The present invention relates to a cardiomyocyte proliferation promoting agent and use thereof. The present invention also relates to a method for screening for a cardiomyocyte proliferation promoting agent and a method for proliferating cardiomyocytes using the cardiomyocyte proliferation promoting agent.

### BACKGROUND ART

Currently, the only radical treatment for drug-resistant heart failure is heart transplantation. In Japan, where aging is rapidly progressing, it is estimated that heart failure patients will continue to increase in the future. However, donors for heart transplantation are extremely scarce, and the development of treatments that can replace heart transplantation is expected. After the report of human iPS cell establishment (Non-Patent Document 1), human iPS cell-derived cardiomyocytes have been a very useful tool as a cell source for treating heart failure, but have many problems in their clinical applications. One of such problems is a low engraftment rate of transplanted cardiomyocytes when cardiomyocytes induced to differentiate from human iPS cells are transplanted.

We transplanted iPS cell-derived cardiomyocytes into immunodeficient mice by intramyocardial injection, and reported that iPS cell-derived cardiomyocytes on day 20 after initiation of differentiation induction had the highest engraftment rate (Non-patent Document 2).

### CITATION LIST

### Non-Patent Documents

Non-Patent Document 1: Takahashi, K. et al. Cell 131, 861-872 (2007).
Non-Patent Document 2: Funakoshi, S. et al. Sci Rep 8, 19111 (2016).

### SUMMARY OF INVENTION

As described above, when cardiomyocytes induced to differentiate from human iPS cells and the like are transplanted, improvement of the engraftment rate of the transplanted cardiomyocytes is a major issue. Accordingly, an object of the present invention is to provide a method for efficiently proliferating cardiomyocytes and increasing the engraftment rate upon transplantation and a reagent to be used therefor.

The present inventors have discovered that iPS cell-derived cardiomyocytes proliferate after post-transplant engraftment, and the degree of proliferation is associated with the cell cycle of transplanted iPS cell-derived cardiomyocytes. As a result of screening for drugs that activate the cell cycle, the present inventors have discovered that drugs such as a retinoic acid receptor agonist, a phosphatidylinositol 3-kinase (PI3K) inhibitor or an isocitrate dehydrogenase 1 (IDH1) inhibitor cause efficient proliferation of cardiomyocytes. The present inventors have further discovered that transplanting cardiomyocytes proliferated using these drugs significantly improves the engraftment rate after transplantation, and thus have completed the present invention.

That is, the present invention is as summarized as follows.

[1] A cardiomyocyte proliferation promoting agent, consisting of a compound having a cell cycle activating ability.
[2] The cardiomyocyte proliferation promoting agent according to [1], wherein the compound having a cell cycle activating ability is a retinoic acid receptor agonist, a phosphatidylinositol 3-kinase (PI3K) inhibitor or an isocitrate dehydrogenase 1 (IDH1) inhibitor.
[3] The cardiomyocyte proliferation promoting agent according to [2], wherein the retinoic acid receptor agonist is AM80.
[4] A method for proliferating cardiomyocytes, comprising a step of culturing cardiomyocytes in a medium comprising the cardiomyocyte proliferation promoting agent according to any one of [1] to [3].
[5] The method for proliferating cardiomyocytes according to [4], wherein the cardiomyocytes are human cardiomyocytes.
[6] The method for proliferating cardiomyocytes according to [4] or [5], wherein the cardiomyocytes are cardiomyocytes differentiated from pluripotent stem cells.
[7] The method for proliferating cardiomyocytes according to [6], wherein the pluripotent stem cells are induced pluripotent stem cells.
[8] Cardiomyocytes, which are proliferated by the method according to any one of [4] to [7] and have improved engraftment ability.
[9] A cardiomyocyte culture kit, comprising cardiomyocytes and the cardiomyocyte proliferation promoting agent according to any one of [1] to [3].
[10] A cardiomyocyte culture medium, comprising the cardiomyocyte proliferation promoting agent according to any one of [1] to [3].
[11] A method for screening for a cardiomyocyte proliferation agent, comprising a step of contacting cardiomyocytes with a test substance in vitro, a step of measuring the cell cycle progression of the cardiomyocytes, and a step of selecting a compound that promotes the cell cycle progression.
[12] The method for screening for a cardiomyocyte proliferation agent according to [11], wherein the cardiomyocytes are cardiomyocytes expressing a gene related to the cell cycle transition from the G1/G0 phase to the S/G2/M phase, and the cell cycle progression is measured by monitoring the expression of the gene.
[13] The method for screening for a cardiomyocyte proliferation agent according to [12], wherein the gene is a Fucci (Fluorescent Ubiquination-based Cell Cycle Indicator) gene.

According to the present invention, a drug that promotes the proliferation of cardiomyocytes can be obtained using cell cycle activation as an index, and cardiomyocytes can be efficiently proliferated by culturing the cardiomyocytes using the thus obtained drug. The obtained cardiomyocytes have an excellent effect such that the engraftment rate is remarkably improved when transplanted, and contribute to regenerative medicine, etc.

### BRIEF DESCRIPTION OF DRAWINGS

FIG. 1 depicts a flow cytometry showing the activation of the cell cycle of cardiomyocytes by AM80 administration.

FIG. 2 depicts a graph showing the digitized results of the flow cytometry in FIG. 1, which is indicated with Control (DMSO) set to 1, N=5 and mean±SD.

FIG. 3 depicts the results of Edu assay showing the activation of the cell cycle of cardiomyocytes by AM80 administration.

FIG. 4 depicts a graph showing the improvement of the engraftment efficiency when the cardiomyocytes cultured with AM80 addition were transplanted.

### DESCRIPTION OF EMBODIMENTS

### <Cardiomyocyte Proliferation Promoting Agent>

The cardiomyocyte proliferation promoting agent of the present invention comprises a compound having a cell cycle activating ability as an active ingredient.

The term “cell cycle activating ability” refers to an effect of promoting the cell cycle progression, and more specifically means an ability of promoting the transition from the G1/G0 phase to the S/G2/M phase.

The expression “promoting the transition from the G1/G0 phase to the S/G2/M phase” refers to acting on cells in the G0 phase or the resting state because of their deviation (escaping) from the cell cycle to undergo transition to the S phase, the G2 phase or the M phase and thus to enter the cell cycle again, or, acting on cells in the G1 phase to undergo cell cycle transition from the G1 phase to the S phase, the G2 phase or the M phase.

The presence or the absence of the “activity to cause the transition from the G0 phase or the G1 phase to the S phase, the G2 phase or the M phase” can be evaluated using, for example, a change in the expression of a gene related to the transition from the G1/G0 phase to the S/G2/M phase as an index. Specifically, an example of a gene related to the transition from the G1/G0 phase to the S/G2/M phase is Fucci, and a change in fluorescence associated with the Fucci transition to the S/G2/M phase can be used as an index. Fucci is disclosed in Cell. 2008 Feb. 8; 132 (3): 487-98. and JP Re-publication of WO2008/114544, and commercially available expression reagents can also be used (Medical & Biological Laboratories, Co., Ltd.).

### <Screening for Cardiomyocyte Proliferation Promoting Agent Using Cell Cycle Activation as an Index>

The method for screening for a cardiomyocyte proliferation promoting agent of the present invention comprises a step of contacting cardiomyocytes with a test substance in vitro, a step of measuring the cell cycle progression of the cardiomyocytes, and a step of selecting a compound that promotes the cell cycle progression.

A more preferred embodiment involves using cardiomyocytes expressing a gene related to the cell cycle transition from the G1/G0 phase to the S/G2/M phase, such as Fucci described above, measuring the cell cycle progression by monitoring the expression of the gene in the cardiomyocytes contacted with drugs, and then selecting a compound that promotes the cell cycle progression.

The Fucci reporter emits orange fluorescence in the G1/G0 phase and emits green fluorescence in the S/G2/M phase of the cell cycle. Since the cell cycle of undifferentiated human iPS cells is activated, most cells emit green fluorescence. However, when human iPS cells are differentiated into cardiomyocytes, the cell cycle usually stops and most cells turn red. Here, when cardiomyocytes are contacted with a drug that activates the cell cycle, the green fluorescence is observed, which is seen in the S/G2/M phase, because of the effect of the drug. A drug that activates the cell cycle can be screened by using the green as an index. For example, with an index such that the proportion of green cells in all cells is 50% or more, preferably 80% or more, a drug can be screened. Further, through comparison of the fluorescence intensity with a case in which no test substance is added, or a case in which a positive control is added, a drug can be screened by using an index such that the proportion of green cells is increased compared to a case in which no test substance is added, or, an index such that the proportion of green cells is at least equivalent to that in a case in which a positive control is added. The fluorescence intensity can be measured using a flow cytometer or the like.

The Fucci gene can be introduced into cardiomyocytes or pre-differentiated pluripotent stem cells by using known vectors such as viral vectors, transposon vectors, plasmid vectors, and appropriate expression promoters.

The fact that the drug activates the cell cycle may be confirmed by examining the activity of incorporation of thymidine analogues, BrdU (5-bromo-2′-deoxyuridine) and EdU (5-ethynyl-2′-deoxyuridine), into cells. For example, BrdU or Edu is added to a cell culture medium together with a candidate drug, and then the amount of BrdU or Edu incorporated into the DNA may be confirmed using a fluorescent labeling substance.

The cardiomyocytes are not particularly limited and may be cardiomyocytes isolated from a living body, but are preferably cardiomyocytes obtained by induction of differentiation of pluripotent stem cells as described below. The cardiomyocytes are preferably characterized by being positive for myocardial markers, cardiac troponin (cTNT or troponin T type 2) and/or aNMC (a myosin heavy chain). The cardiomyocytes may include myocardial progenitor cells as long as they express these markers.

In the screening method of the present invention, any test substance can be used. Examples of test substances include cell extracts, cell culture supernatants, microbial fermentation products, marine organism-derived extracts, plant extracts, purified and partially purified proteins, peptides, non-peptide compounds, synthetic low molecular weight compounds, and natural compounds. The test substances can be obtained using many techniques of known combinatorial library methods such as 1) a biological library method, 2) a synthetic library method using deconvolution, 3) a 1 bead 1 compound library method, and 4) a synthetic library method using affinity chromatography selection, etc.

A test substance can be contacted with cardiomyocytes by adding the test substance to a medium at an appropriate concentration under normal culture conditions for cardiomyocytes, and then incubating for 1 to 3 hours, for example. When cardiomyocytes differentiated from pluripotent stem cells are used, a test substance is preferably contacted with cardiomyocytes in a sufficiently differentiated state, such that the cardiomyocytes sufficiently express a myocardial marker, for example, cardiomyocytes 15 to 25 days after initiation of differentiation induction.

### <Specific Examples of Cardiomyocyte Proliferation Agents>

There are no particular limitations on the type of cardiomyocyte proliferation promoting agent comprising a compound having a cell cycle activating ability as an active ingredient. Specific examples thereof include a retinoic acid receptor agonist, a phosphatidylinositol 3-kinase (PI3K) inhibitor or an isocitrate dehydrogenase 1 (IDH1) inhibitor.

### <Retinoic Acid Receptor Agonist>

The retinoic acid receptor agonist is not particularly limited as long as it is a compound (excluding retinoic acid) that can bind to the retinoic acid receptor (RAR) and activate its signaling pathway. Specific examples thereof include AM80 (Tamivaloten), LGD1550, E6060, AM580 (CD336), AGN193312, AM555S, CD2314, AGN193174, LE540, CD437, CD666, CD2325, SR11254, SR11363, SR11364, AGN193078, TTNN (Ro19-0645), CD270, CD271, CD2665, SR3985, AGN193273, CH55, 2AGN190521, CD2366, AGN193109, Re80, Ro40-6976, Ro13-7410 (TTNPB), Ro11-0874, Ro04-3780 (13-cis-RA), Ro11-4824 (4-oxo-RA), Ro11-1813, Ro08-8717, Ro10-0191, Ro10-2655 (4-hydroxy-RA), Ro11-0976, Ro40-6055, Ro41-5253, and CD2019.

### <PI3K Inhibitor>

Examples of the PI3K inhibitor include the following compounds.

Direct inhibitors of PI3K: for example, Wortmannin, LY294002, AS605240, ZSTK474, PIK-75 Hydrochloride, IPI-145 (INK1197), GDC-0941, CAL-101 (Idelalisib, GS-1101), BEZ235 (NVP-BEZ235, Dactolisib), BKM120 (NVP-BKM120, Buparlisib), GSK2636771, CZC24832, GDC-0032, VS-5584 (SB2343), TG100713, BYL719, CUDC-907, 3-Methyladenine, YM201636, BGT226 (NVP-BGT226), BAY80-6946 (Copanlisib), PF-04691502, PKI-402, CH5132799, GDC-0980 (RG7422), NU7441 (KU-57788), AS-252424, AS-604850, CAY10505, GSK2126458 (GSK458), A66, PF-05212384 (PKI-587), Palomid529 (P529), PIK-294, PIK-293, SAR245409 (XL765), PIK-93, AZD6482, AS-605240, GSK1059615, TG100-115, IC-87114, PIK-75, PIK-90, TGX-221, XL147, PI-103, IC486068
PI3K-Akt pathway inhibitors: for example, MK-2206, GSK690693, GDC-0068, A-674563, and CCT128930
PI3K-PDK1 pathway inhibitors: for example, BX-912, BX-795, OSU-03012, and PHT-427

### <IDH1 Inhibitor>

Examples of IDH1 inhibitors include, but are not limited to, AGI5198 (N-[2-(cyclohexylamino)-1-(2-methylphenyl)-2-oxoethyl]-N-(3-fluorophenyl)-2-methyl-1H-imidazole-1-acetamide), AG-120 (Agios Pharmaceuticals, Inc.), IDH-C227 (Agios Pharmaceuticals, Inc.), and ML309 (Agios Pharmaceuticals, Inc.). The IDH1 inhibitor may also be an IDH inhibitor disclosed in any of the following patent publications: WO2014062511; WO2012171506; WO2012171337; WO2013107405; WO2013107291; WO2012009678; and WO2011072174.

### <Method for Proliferating Cardiomyocytes>

The method for proliferating cardiomyocytes of the present invention comprises a step of culturing cardiomyocytes in a medium comprising a cardiomyocyte proliferation promoting agent.

Here, cardiomyocytes are characterized by being positive for myocardial markers, cardiac troponin (cTNT or troponin T type 2) and/or αMHC (α myosin heavy chain). Cardiomyocytes may include myocardial progenitor cells.

The cardiomyocytes may be cardiomyocytes isolated from a living body, but are preferably cardiomyocytes induced to differentiate from pluripotent stem cells.

### <Pluripotent Stem Cells>

Pluripotent stem cells are stem cells having pluripotency such that these cells can be differentiated into many cells present in a living body and also having proliferative capacity, and include any cell that is induced to differentiate into the primitive endoderm. Example of pluripotent stem cells are not particularly limited, and include embryonic stem (ES) cells, induced pluripotent stem (iPS) cells, embryonic stem (ntES) cells derived from a clone embryo obtained by nuclear transplantation, sperm stem cells (“GS cells”), embryonic germ cells (“EG cells”), cultured fibroblasts, and pluripotent cells (Muse cells) derived from bone marrow stem cells. Preferred pluripotent stem cells are iPS cells and ES cells. Pluripotent stem cells are preferably derived from mammals, more preferably derived from primates, and even more preferably derived from humans.

Methods for producing iPS cells are known in the art, and can be produced by introducing reprogramming factors into any somatic cells. Here, examples of the reprogramming factor include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall1, Sall4, Esrrb, Nr5a2, Tbx3 and Glis1 genes or gene products thereof. These reprogramming factors may be used alone or in combination. Examples of combinations of reprogramming factors include those described in WO2007/069666, WO2008/118820, WO2009/007852, WO2009/032194, WO2009/058413, WO2009/057831, WO2009/075119, WO2009/079007, WO2009/091659, WO2009/101084, WO2009/101407, WO2009/102983, WO2009/114949, WO2009/117439, WO2009/126250, WO2009/126251, WO2009/126655, WO2009/157593, WO2010/009015, WO2010/033906, WO2010/033920, WO2010/042800, WO2010/050626, WO2010/056831, WO2010/068955, WO2010/098419, WO2010/102267, WO2010/111409, WO2010/111422, WO2010/115050, WO2010/124290, WO2010/147395, WO2010/147612, Huangfu D, et al. (2008), Nat. Biotechnol. 26: 795-797, Shi Y, et al. (2008), Cell Stem Cell, 2: 525-528, Eminli S, et al. (2008), Stem Cells. 26: 2467-2474, Huangfu D, et al. (2008), Nat. Biotechnol. 26: 1269-1275, Shi Y, et al. (2008), Cell Stem Cell, 3, 568-574, Zhao Y, et al. (2008), Cell Stem Cell, 3: 475-479, Marson A, (2008), Cell Stem Cell, 3, 132-135, Feng B, et al. (2009), Nat. Cell Biol. 11: 197-203, R. L. Judson et al., (2009), Nat. Biotechnol., 27: 459-461, Lyssiotis C A, et al. (2009), Proc Natl Acad Sci U.S.A. 106: 8912-8917, Kim J B, et al. (2009), Nature. 461: 649-643, Ichida J K, et al. (2009), Cell Stem Cell. 5: 491-503, Heng J C, et al. (2010), Cell Stem Cell. 6: 167-74, Han J, et al. (2010), Nature. 463: 1096-100, Mali P, et al. (2010), Stem Cells. 28: 713-720, Maekawa M, et al. (2011), and Nature. 474: 225-9.

Examples of somatic cells to be used for preparation of iPS cells include fetal (pup) somatic cells, neonatal (pup) somatic cells, and mature healthy or diseased somatic cells, as well as any of primary cultured cells, passaged cells, and established cell lines. Specific examples of somatic cells include (1) tissue stem cells (somatic stem cells) such as neural stem cells, hematopoietic stem cells, mesenchymal stem cells, and dental pulp stem cells, (2) tissue progenitor cells, (3) differentiated cells such as blood cells (peripheral blood cells, cord blood cells, etc.), lymphocytes, epithelial cells, endothelial cells, muscle cells, fibroblasts (skin cells, etc.), hair cells, hepatocytes, gastric mucosa cells, intestinal cells, spleen cells, pancreatic cells (pancreatic exocrine cells), brain cells, lung cells, kidney cells and fat cells.

Examples of methods for inducing differentiation from pluripotent stem cells to cardiomyocytes include methods described in the following documents.

Funakoshi, S. et al. Sci Rep 8, 19111 (2016) Miki, K. et al. Cell Stem Cell. 2015 Jun. 4; 16 (6): 699-711

Laflamme M A & Murry C E, Nature 2011, May 19; 473 (7347): 326-35 Review

Other than these methods, examples thereof include, but are not particularly limited to, a method for producing cardiomyocytes by forming a cell mass (embryoid body) by suspension culture of induced pluripotent stem cells (WO2016/104614), a method for producing cardiomyocytes in the presence of a substance that suppresses BMP signaling (WO2005/033298), a method for producing cardiomyocytes by adding Activin A and BMP in order (WO2007/002136), a method for producing cardiomyocytes in the presence of a substance that promotes the activation of the canonical Wnt signaling pathway (WO2007/126077), and a method for producing cardiomyocytes in the presence of cyclosporin A after isolation of Flk/KDR positive cells from induced pluripotent stem cells (WO2009/118928).

The step of culturing cardiomyocytes in a medium comprising the cardiomyocyte proliferation agent of the present invention can be performed by dissolving the cardiomyocyte proliferation agent at an appropriate concentration in an aqueous or non-aqueous solvent, adding the cardiomyocyte proliferation agent with a concentration that enables exertion of a proliferative effect in an appropriate medium (e.g., a minimal essential medium (MEM) containing about 5 to 20% fetal calf serum, Dulbecco's modified Eagle medium (DMEM), alpha-MEM, RPMI1640 medium, 199 medium, and F12 medium, etc), and then culturing cardiomyocytes for a certain period. The concentration of the cardiomyocyte proliferation agent varies depending on the type of a substance used. For example, in the case of a retinoic acid receptor agonist such as Am80, the concentration ranges from preferably 10 nM to 10 μM, more preferably 100 nM to 5 μM, and in the case of a PI3K inhibitor, the concentration ranges from preferably 10 nM to 10 μM, and more preferably 100 nM to 5 μM. In the case of an IDH1 inhibitor, the concentration preferably ranges from 5 nM to 5 μM, and more preferably 50 nM to 2 μM. The culture period is not particularly limited as long as it is a sufficient time for target cells to proliferate. For example, the culture period is appropriately selected from the range of 1 to 10 days. When cardiomyocytes induced to differentiate from pluripotent stem cells are proliferated, the cardiomyocyte proliferation agent may be added during the differentiation step, if the cardiomyocytes already exist. For example, the cardiomyocyte proliferation agent may be added on days 15 to 25 after the initiation of differentiation induction.

Cardiomyocytes proliferated using the cardiomyocyte proliferation agent of the present invention have an excellent property of exhibiting improved engraftment ability when transplanted to the myocardial tissue, and thus are suitably used as a cell preparation for transplantation into a patient who requires transplantation of cardiomyocytes. Examples of patients who require transplantation of cardiomyocytes include, but are not limited to, patients with diseases caused by myocardial cell defects such as myocarditis, myocardial infarction and myocardial damage. The amount of cells to be transplanted is appropriately selected depending on the type and degree of the disease, and the number of transplantations can be one or more. The method of transplantation is not limited, and may be injection to a disease site, or a cardiomyocyte sheet may be prepared and applied to the disease site.

The present invention also provides a cardiomyocyte culture kit comprising cardiomyocytes and the cardiomyocyte proliferation promoting agent. The cardiomyocyte culture kit can include instructions for use describing methods for handling the cardiomyocyte proliferation promoting agent and culturing.

The present invention also provides a cardiomyocyte culture medium comprising the cardiomyocyte proliferation promoting agent. As the medium, a general medium containing components necessary for culturing cardiomyocytes can be used. The cardiomyocyte proliferation promoting agent may be added in advance to the medium, or may be prepared separately from the medium so that it is added at the time of use. The medium for cardiomyocytes may be a medium for differentiation of pluripotent stem cells into cardiomyocytes.

### EXAMPLES

Hereinafter, the present invention is more specifically described with reference to Examples, but the embodiment of the present invention is not limited to the following Examples.

### <Screening for Cardiomyocyte Proliferation Agent Based on Cell Cycle Activation>

The Fucci gene (Cell. 2008 Feb. 8; 132 (3): 487-98.) was constitutively expressed under the CAG promoter using the PiggyBac transposon vector system (System Biosciences) in a healthy human-derived iPS cell line.

The Fucci gene-introduced iPS cell line was induced to differentiate into cardiomyocytes by the embryoid body method described in Funakoshi, S. et al. Sci Rep 8, 19111 (2016), cardiomyocytes were extracted using a cell sorter on day 20 after induction, and then seeded into a 384-well plate at 2500 cells per well.

On day 22 after differentiation induction, about 4000 types of compounds were administered, and on day 25, the reactivity of the proliferative capacity of iPS cell-derived cardiomyocytes to the drugs was evaluated based on the intensity of Fucci's green fluorescence and the number of cells.

The Fucci reporter emits orange fluorescence in the G1/G0 phase and green fluorescence in the S/G2/M phase of the cell cycle. Since the cell cycle of undifferentiated human iPS cells is activated, most cells emit green fluorescence. However, when these iPS cells are differentiated into cardiomyocytes, the cell cycle usually stops and most cells turn red. However, it was revealed that when some compounds are administered, cell cycle activation takes place even in the differentiated state, and the Fucci reporter emits green fluorescence.

Specifically, as a result of screening, three types of compounds that promote the proliferation and cell division of iPS cell-derived cardiomyocytes (retinoic acid receptor (RAR) agonists (All trans retinoic acid, CH55, AM80, AM580, CD437), PI3K inhibitors (Wortmannin, CAY10505, CZC24832), and an IDH inhibitor (AGI5198)) were identified.

### <Evaluation of AM80—Cell Cycle Activation and Proliferative Capacity of Cardiomyocytes>

Among the obtained compounds, further experiments were performed on AM80.

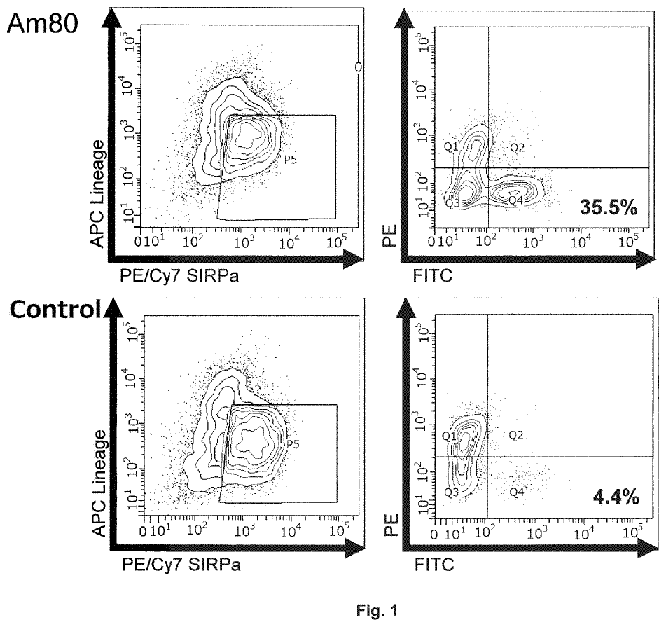

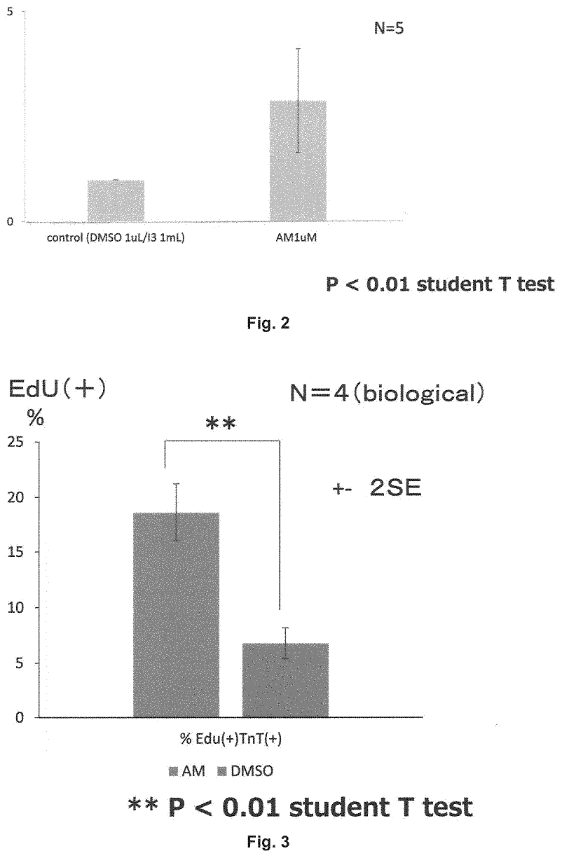

1 μM AM80 was administered to cardiomyocytes on day 18 of differentiation induction and then analysis was conducted by flow cytometry 48 hours later. As shown in FIG. 1 and FIG. 2, a significant increase was observed in green fluorescent cells, confirming the activation of cell cycle.

Further, human iPS cell-derived cardiomyocytes on day 18 after differentiation induction were treated with AM80 (1 μM) for 48 hours, and cell proliferation analysis was performed using EdU assay. As a result, as shown in FIG. 3, a significant increase in cell proliferation activity was observed in the AM80-treated group compared to the control group (DMSO-treated group). For the EdU assay, Click-it EdU Pacific Blue flow cytometry assay kit, catalog number C10418 (Invitrogen) was used.

### <Evaluation of AM80-Engraftment Rate of Transplanted Cardiomyocytes>

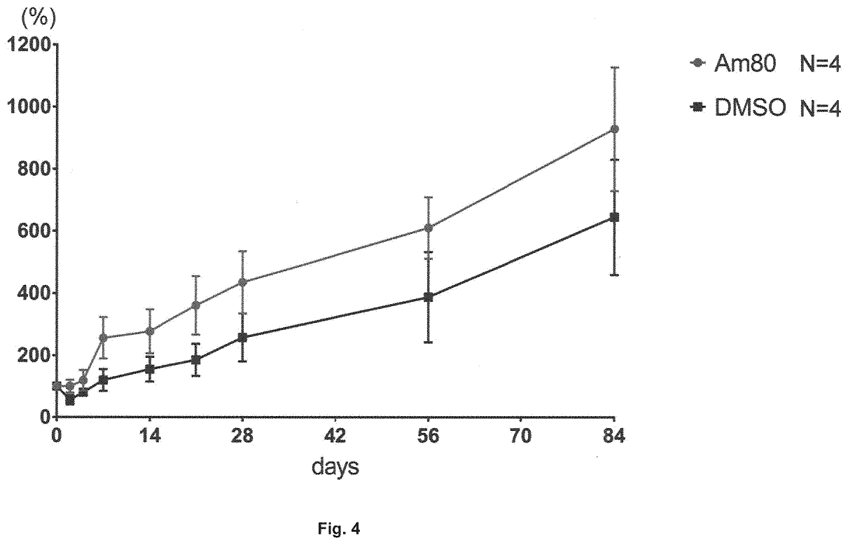

Cardiomyocytes were induced to differentiate from iPS cells (Funakoshi, S. et al. Sci Rep 8, 19111 (2016)) derived from a healthy individual, which constantly express luciferase, by the method described in this document. The cardiomyocytes were treated with AM80 (1 μM) for 48 hours from day 18 after differentiation induction. Cardiomyocytes were extracted by flow cytometry, and then transplanted (1000,000 cells) into a myocardial infarction model immunodeficient mouse (NRG mouse with the left anterior descending coronary artery ligated) by intramyocardial injection. The evaluation of the engraftment efficiency of cardiomyocytes transplanted by in vivo optical imaging (IVIS) revealed significant improvement in engraftment efficiency compared to the control group (DMSO treatment group), as shown in FIG. 4. This demonstrated for the first time that the engraftment efficiency is improved by AM80 in an in vivo animal model.

From these results, drugs such as AM80 activate the cell cycle of cardiomyocytes, and transplantation of cells with activated cell cycle is expected to realize myocardial regeneration therapy with high engraftment efficiency.

1. A cardiomyocyte proliferation promoting agent, consisting of a compound having a cell cycle activating ability.

2. The cardiomyocyte proliferation promoting agent according to claim 1, wherein the compound having a cell cycle activating ability is a retinoic acid receptor agonist, a phosphatidylinositol 3-kinase (PI3K) inhibitor or an isocitrate dehydrogenase 1 (IDH1) inhibitor.

3. The cardiomyocyte proliferation promoting agent according to claim 2, wherein the retinoic acid receptor agonist is AM80.

4. A method for proliferating cardiomyocytes, comprising a step of culturing cardiomyocytes in a medium comprising a compound having a cell cycle activating ability.

5. The method for proliferating cardiomyocytes according to claim 4, wherein the cardiomyocytes are human cardiomyocytes.

6. The method for proliferating cardiomyocytes according to claim 4, wherein the cardiomyocytes are cardiomyocytes differentiated from pluripotent stem cells.

7. The method for proliferating cardiomyocytes according to claim 6, wherein the pluripotent stem cells are induced pluripotent stem cells.

8. Cardiomyocytes, which are proliferated by the method according to claim 4 and have improved engraftment ability.

9. A cardiomyocyte culture kit, comprising cardiomyocytes and the cardiomyocyte proliferation promoting agent according to claim 1.

10. A cardiomyocyte culture medium, comprising the cardiomyocyte proliferation promoting agent according to claim 1.

11. A method for screening for a cardiomyocyte proliferation promoting agent, comprising a step of contacting cardiomyocytes with a test substance in vitro, a step of measuring the cell cycle progression of the cardiomyocytes, and a step of selecting a compound that promotes the cell cycle progression.

12. The method for screening for a cardiomyocyte proliferation promoting agent according to claim 11, wherein the cardiomyocytes are cardiomyocytes expressing a gene related to the cell cycle transition from the G1/G0 phase to the S/G2/M phase, and the cell cycle progression is measured by monitoring the expression of the gene.

13. The method for screening for a cardiomyocyte proliferation promoting agent according to claim 12, wherein the gene is a Fucci (Fluorescent Ubiquination-based Cell Cycle Indicator) gene.

14. The method for proliferating cardiomyocytes according to claim 4, wherein the compound having a cell cycle activating ability is a retinoic acid receptor agonist, a phosphatidylinositol 3-kinase (PI3K) inhibitor or an isocitrate dehydrogenase 1 (IDH1) inhibitor.

15. The method for proliferating cardiomyocytes according to claim 14, wherein the retinoic acid receptor agonist is AM80.
